(19) United States
(12) Patent Application Publication
(10) Pub. No.: US 2018/0038929 A1
(43) Pub. Date: Feb. 8, 2018

(54) METHOD AND APPARATUS FOR SETTING AND/OR ADJUSTING SCAN PARAMETERS FOR A SCAN SEQUENCE OF A MAGNETIC RESONANCE EXAMINATION

(71) Applicant: Siemens Healthcare GmbH, Erlangen (DE)

(72) Inventors: Friedemann Paulini, Oberasbach (DE); Marcus Wuebbe, Herzogenaurach (DE)

(73) Assignee: Siemens Healthcare GmbH, Erlangen (DE)

(21) Appl. No.: 15/668,748

(22) Filed: Aug. 4, 2017

(30) Foreign Application Priority Data
Aug. 5, 2016 (DE) 102016214608.4

(51) Int. Cl. G01R 33/54; A61B 5/055

(52) U.S. Cl. CPC G01R 33/543; G01R 33/546; A61B 5/0555

(57) ABSTRACT

In a method and apparatus for setting and/or adjusting scan parameters for a scan sequence of a magnetic resonance examination, manual selection of a scan parameter to be set is made, followed by setting and/or changing a value of the selected scan parameter to be set. At least one dependent scan parameter is determined in a computer, the value of which, on a change to a value of the scan parameter to be set, would undergo a change provided that existing image-acquisition criteria are retained. The computer also determines a suggested change for a change to the value of the at least one dependent scan parameter, and displays the at least one dependent scan parameter and the suggested change for a change to the value of the at least one dependent scan parameter.

### BACKGROUND OF THE INVENTION

### Field of the Invention

The present invention concerns a method for setting and/or adjusting scan parameters for a scan sequence of a magnetic resonance examination, and a magnetic resonance apparatus configured to implement such a method.

### Description of the Prior Art

To be able to start a magnetic resonance examination of a patient, it is first necessary for an operator, in particular a medical technician, to select the appropriate magnetic resonance protocols and/or scan sequences. Such scan protocols and/or scan sequences have a number of scan parameters that may be set and/or adjusted, and/or must be set and/or adjusted by the operator. When individual scan parameters are set and/or adjusted, this can result in further changes to further scan parameters in order, for example, to retain a constant scan time and/or a constant image quality for the upcoming magnetic resonance examination. These dependences are not always evident to the operator and can also result in changes to scan parameters that the operator has previously changed or set to a specific value.

### SUMMARY OF THE INVENTION

An object of the present invention is to provide transparency for an operator when scan parameters are set and/or adjusted, i.e., are entered into the control computer.

This object is achieved in accordance with the invention by a method for setting and/or adjusting (entering) scan parameters for a scan sequence of a magnetic resonance examination with the following steps.

A manual selection is made of a scan parameter to be set, followed by setting and/or changing a value of the selected scan parameter. In a processor to which the set or changed scan parameter is provided, a determination is made of at least one dependent scan parameter, the value of which, on a change to a value of the selected scan parameter, would undergo a change that still causes existing image-acquisition criteria to be retained. The processor further makes determination of a suggested change for a change to the value of the at least one dependent scan parameter and causes the at least one dependent scan parameter, and the suggested change for a change to the value of the at least one dependent scan parameter, to be displayed.

In this context, a scan parameter means a parameter that is sequence-specific and can be adjusted and/or set by an operator, such as and echo time and/or slice thickness and/or slice orientation, etc. For example, an operator can influence image quality and/or scan time by the skillful selection and/or adjustment of the scan parameters.

For example, the scan parameters can be used to influence scan time and/or image quality and/or an image resolution. If it is required to retain the highest possible image quality and/or the shortest possible scan time and/or the highest possible image quality on a change and/or adjustment of a selected scan parameter, this necessitates a change to and/or adjustment of further scan parameters, in particular those dependent upon the selected scan parameter.

The scan parameter to be set is selected by the operator, in particular by a medical technician. It is also possible for the value of the scan parameter to be set to be set or changed manually by the operator.

The determination of the at least one dependent scan parameter and the determination of the suggested change are performed automatically by a control computer and/or a computing processor of the magnetic resonance apparatus. To this end, the control computer, computing processor include the necessary computer programs and/or software that are executed in order to implement the determination of at least one dependent scan parameter and the determination of the suggested change. The display of the at least one dependent scan parameter takes place at a display monitor in communication with the control computer or the computing processor.

The suggested change preferably represents values for the dependent scan parameters that these scan parameters would adopt upon a change to the value of the selected scan parameter to be set, if the image-acquisition criteria are kept constant. The image-acquisition criteria can, for example, comprise image quality and/or scan time and/or image resolution and/or further image-acquisition criteria that are considered advisable to those skilled in the art.

The invention achieves transparency for the operator when scan parameters are set and/or adjusted when adjusting scan sequences. All dependent changes that result in a change to a selected scan parameter to be set can be displayed to the operator. This makes it easier for inexperienced and/or untrained operators to adjust and/or change scan parameters. It is advantageously possible for the suggested change to be displayed to the operator directly when the selected scan parameter is set or adjusted so that all changes associated therewith are immediately evident to the operator.

In an embodiment of the invention, the at least one dependent scan parameter is a fixed scan parameter. In this context, a fixed scan parameter means a parameter of a scan sequence, the value of which is a fixed value independently of further settings and/or changes to further scan parameters, in particular when the selected scan parameter to be set is changed and/or set. Hence, the fixed scan parameter retains its fixed value unchanged, in accordance with the invention, the fixed scan parameter is not included in the suggested change for a change to the value of the at least one dependent scan parameter. This enables the operator to define and/or specify individual scan parameters, which should always have the same value for each scan. Preferably, a scan parameter is defined and/or specified as a fixed scan parameter by the operator.

In another embodiment, there are at least two dependent scan parameters, wherein a first of the at least two dependent scan parameters is the fixed scan parameter, and the suggested change is a change to values of at least a further one of the at least two dependent scan parameters. This makes it simple to take account of a scan parameter defined by the operator as a fixed scan parameter when a suggested change is set. This allows the image-acquisition criteria, such as high image quality and/or short scan time, to be retained and/or kept constant and taken into account in the creation of the suggested change. Preferably, a value of the fixed scan parameter is unaffected by the creation of the suggested change. The fixed scan parameter is taken into account in the creation of the suggested change automatically by the control computer so that no additional action on the part of the operator is required.

In a further embodiment of the invention, the suggested change for setting the scan parameter is a change to the image-acquisition criterion if a change to the values of the dependent scan parameters is not possible. A change to the values of dependent scan parameters can be, for example, no longer possible when these values already have a maximum permissible limit value and a further change to the value would result in a value outside a permissible setting range for this dependent scan parameter. A change to the image-acquisition criteria can enable scan parameters to be retained, which have already been defined as fixed scan parameters by the operator. Moreover, it is possible to achieve a wide range of variation for further dependent scan parameters that are not included in the fixed scan parameters.

In another embodiment, the suggested change represents an adjustment of and/or change to values of scan parameters that have already been adjusted or changed. This enables previously defined image-acquisition criteria to be retained. Hence, values of dependent scan parameters specified as fixed scan parameters can also be retained,

The invention concerns a magnetic resonance apparatus with a data acquisition scanner operated by a control computer and a user interface, wherein the magnetic resonance apparatus is designed to implement the inventive method for setting and/or adjusting scan parameters for a scan sequence of a magnetic resonance examination.

The inventive magnetic apparatus enables transparency to be achieved for the operator, in particular a medical technician, when scan parameters are set and/or adjusted when scan sequences are adjusted. All dependent changes that would result in a change to a selected scan parameter to be set can be displayed to the operator. This makes it easier for inexperienced and/or untrained operators to adjust and/or change scan parameters. The suggested change can be displayed to the operator directly in the setting of the scan parameter so that all changes associated therewith are immediately evident to the operator.

The advantages of the magnetic resonance apparatus according to the invention correspond to the advantages of the method according to the invention for setting and/or adjusting scan parameters for a scan sequence of a magnetic resonance examination, which were explained above in detail. Features, advantages or alternative embodiments of the method are also applicable to the apparatus.

The present invention also encompasses a non-transitory, computer-readable data storage medium encoded with programming instructions (program code) that, when the storage medium is loaded into a computer system of a magnetic resonance apparatus, cause the computer system to operate the magnetic resonance apparatus in order to implement any or all embodiments of the method according to the invention, as described above. The program code is formulated as software, and may be a source code, which still must be compiled and linked, or which must be interpreted, or may be executable software code that only needs to be loaded into the computer system in order to be executed.

### BRIEF DESCRIPTION OF THE DRAWINGS

FIG. 1 schematically illustrates a magnetic resonance apparatus in accordance with the invention.

FIG. 2 is a flowchart of the method according to the invention for setting and/or adjusting scan parameters for a scan sequence of a magnetic resonance examination.

FIG. 3 shows an exemplary screen display of dependent scan parameters in accordance with the invention.

### DESCRIPTION OF THE PREFERRED EMBODIMENTS

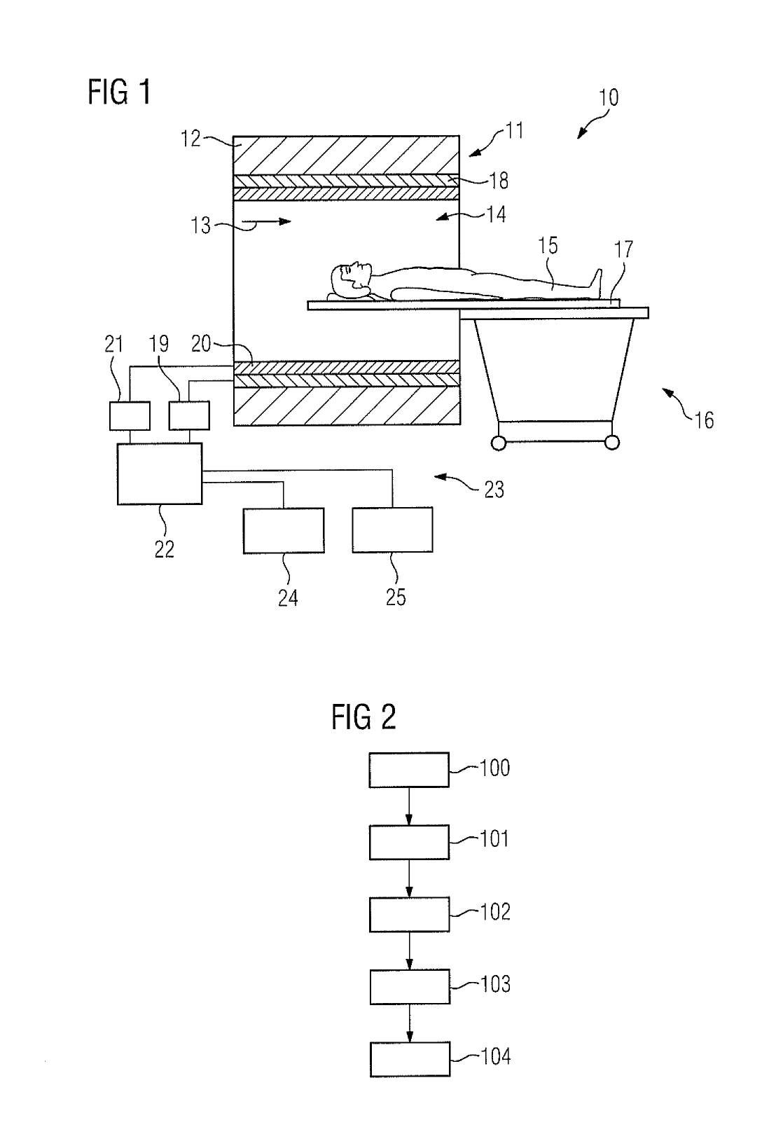

FIG. 1 is a schematic depiction of a magnetic resonance apparatus 10. The magnetic resonance apparatus 10 has and MR data acquisition scanner 11 with a superconducting basic field magnet 12 that generates a strong and constant basic magnetic field 13. The scanner 11 has a patient receiving area 14 for receiving a patient. In the exemplary embodiment, the patient receiving area 14 has a cylindrical shape and is surrounded in a circumferential direction by the scanner 11. An embodiment of the patient receiving area 14 deviating from the exemplary embodiment is also conceivable. The patient 15 can be moved into the patient receiving area 14 by means of a patient support 16. The patient support device 16 has a patient table 17 that is movable within the patient receiving area 14.

The scanner 11 further has a gradient coil arrangement 18 that generates magnetic field gradients that are used for spatially encoding MR signals during imaging. The gradient coil arrangement 18 is controlled by a gradient controller 19 of the magnetic resonance apparatus 10. The scanner 11 further has a radio-frequency (RF) antenna 20 controlled by a radio-frequency controller 21 so as to radiate radio-frequency pulse sequence into an examination volume substantially formed by the patient-receiving area 14 of the scanner 11. The radio-frequency pulse sequence causes certain nuclear spins in the patient 15 to deviate from the magnetic field lines of the basic magnetic field by a specified amount known as a flip angle. As these excited nuclear spins return to the steady state, they emit the aforementioned magnetic resonance signals, which are received by the same antenna from which the RF pulses were radiated, or by a different antenna.

To control the basic field magnet 12, the gradient controller 19 and the radio-frequency controller 21, the magnetic resonance apparatus 10 has a control computer 22. The control computer 22 controls the magnetic resonance apparatus 10 centrally, such as for the performance of a predetermined imaging gradient echo sequence. The control computer 22 also has an evaluation processor (not separately shown) for evaluating medical image data acquired during the magnetic resonance examination. The magnetic resonance apparatus 10 has a user interface 23 that is connected to the control computer 22. Control information such as imaging parameters, and reconstructed magnetic resonance images, can be displayed to a medical operator on a display unit 24, for example on at least one monitor, of the user interface 23. The user interface 23 also has an input unit 25 via which information and/or parameters can be entered by the medical operator during a scanning process.

The control computer 22 is also configured to implement the method for setting and/or adjusting scan parameters for a scan sequence of a magnetic resonance examination in accordance with the invention. To this end, the control computer 22 comprises computer programs and/or software that can be loaded directly in a memory of the control computer 22, with program code for implementing the method for setting and/or adjusting scan parameters for a scan sequence of a magnetic resonance examination, when the computer programs and/or software are executed in the control computer 22. To this end, the control computer 22 has a processor configured to execute the computer programs and/or software. Alternatively, the computer programs and/or software can be stored on an electronically readable data medium embodied separately from the control computer 22.

The method for setting and/or adjusting scan parameters for a scan sequence of a magnetic resonance examination is shown in more detail in FIG. 2. In a first method step 100, manual selection of a scan parameter to be set for a scan sequence is performed. The manual selection is effected by the operator, such as a medical technician. The selection can be made from a number of scan parameters that are to be set and/or can be adjusted for a scan sequence.

The manual selection is performed via the user interface 23, wherein the operator can use the user interface 23 to access scan parameters of the individual scan sequences that are to be set and/or can be adjusted. Alternatively, the manual selection can also be performed by an external user interface. The user interface 23, in particular the display unit 24 of the user interface 23, is used to display to the user the number of scan parameters that are to be set and/or can be adjusted.

In a further method step 101, a value of the scan parameter to be set is set and/or changed. The setting and/or changing of the value of the scan parameter to be set is performed manually by the operator using the user interface 23.

As soon as a value of the scan parameter to be set has been set and/or changed, in a further method step 102 a determination of at least one dependent scan parameter is performed automatically. In order to keep existing image-acquisition criteria, such as in particular high image quality and/or short scan time, constant and/or at the same value, a value of the dependent scan parameters undergoes a change when the scan parameters to be set are changed and/or adjusted.

The determination of the at least one dependent scan parameter is performed automatically by the control computer 22. In addition to the determination of the at least one dependent scan parameter, a determination of a suggested change for a change of a value of the at least one dependent scan parameter is also performed automatically by the control computer 22 in a further method step 103. The suggested value for the at least one dependent scan parameter preferably corresponds to the value of the dependent scan parameter that the latter would adopt if the value of the scan parameters to be set is changed and the image-acquisition criteria are kept constant and/or stay the same.

Typically, a number of dependent scan parameters are determined, the values of which change when the value of the scan parameter to be set is set and/or changed. The number of dependent scan parameters can also include a fixed scan parameter, the value of which has already been specified at a fixed value by the user and hence is not available for changing when the parameters for the scan sequence are set. The specification of a fixed scan parameter can be performed before the method step 100 of the manual selection of a scan parameter to be set.

If there are two or more dependent scan parameters and the two or more dependent scan parameters comprise the fixed scan parameter, the suggested change can contain a change to the values of the dependent scan parameters that are not represented by the fixed scan parameter. The control computer 22 attempts to achieve the image-acquisition criteria, such as high image quality and/or short scan time by changing the values of the further dependent scan parameters.

If a change to the dependent scan parameters for retaining the specified image-acquisition criteria is not possible in the method step 103 of the determination of the suggested change, the suggested change can also comprise a change to the image-acquisition criteria.

Similarly, in the method step of the determination of the suggested change, the suggested change can also be a change to the image-acquisition criteria if a fixed scan parameter is present and a change to the further dependent scan parameters to retain the specified image-acquisition criteria is not possible. A change to the values of dependent scan parameters may no longer be possible, for example, if these values already include a maximum permissible limit value and a further change to the value would result in a value lying outside a permissible setting range for this dependent scan parameter.

In addition, in the method step 103 of the determination of the suggested change, the suggested change can also comprise a change to scan parameters that have already been set and/or adjusted if a change to the dependent scan parameters to retain the specified image-acquisition criteria is not possible. Similarly, in the method step of the determination of the suggested change, the suggested change can also comprise a change to the image-acquisition criteria if a fixed scan parameter is present and a change to the further dependent scan parameters to retain the specified image-acquisition criteria is not possible.

Following a determination of the suggested change, in a further method step 104, the at least one dependent scan parameter and the suggested change for a value of the at least one dependent scan parameter are displayed. The displaying is performed by the display unit 24 of the user interface 23.

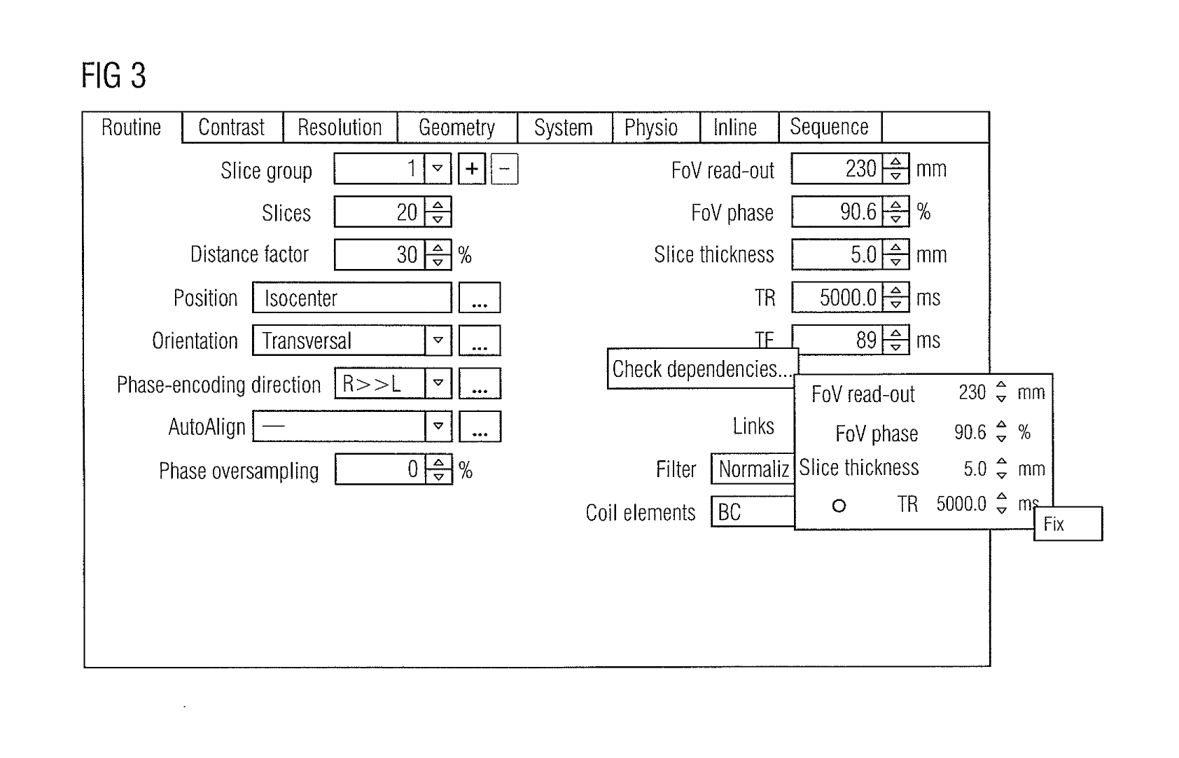

FIG. 3 shows an exemplary display for a suggested change. In this exemplary embodiment, the scan parameter to be set comprises an echo time (TE). The dependent scan parameters are displayed in an extra display window. In the present exemplary embodiment, the dependent scan parameters comprise a FoV read-out, a FoV Phase, a slice thickness and a repetition time (TR). The display also shows that the repetition time is a fixed scan parameter in that this information is depicted in an extra window next to the echo time. In addition, as well the display showing the dependent parameters, the changed and/or adjusted values thereof are also displayed.

With a specification of the change and/or setting of the value of the scan parameter, the user accepts the suggested change for setting and/or changing the dependent scan parameters.

Although modifications and changes may be suggested by those skilled in the art, it is the intention of the Applicant to embody within the patent warranted hereon all changes and modifications as reasonably and properly come within the scope of the Applicant's contribution to the art.

1. A method for setting and/or adjusting at least one scan parameter of a scan sequence in order to conduct a magnetic resonance examination with the scan sequence, said method comprising:
making a manual selection entry into a computer that designates said at least one scan parameter to be set;
making a further entry into the computer that sets and/or changes a value of the at least one selected scan parameter;
in said computer, determining at least one dependent scan parameter in said scan sequence that has a value which, due to a change of the value of the selected scan parameter to be set, would undergo a change under a constraint that existing image-acquisition criteria are retained;
in said computer, determining a suggested change to the value of said at least one dependent scan parameter; and
at a display in communication with said computer, displaying said at least one dependent scan parameter and the suggested change.

2. A method as claimed in claim 1 wherein said at least one dependent scan parameter is a fixed scan parameter of said scan sequence.

3. A method as claimed in claim 2 comprising, in said computer, determining at least two dependent scan parameters in said scan sequence with a first of said at least two dependent scan parameters being a fixed scan parameter, and wherein said suggested change is a change to a value of at least a further one of said at least two dependent scan parameters.

4. A method as claimed in claim 2 comprising determining said suggested change as a change to the image acquisition criteria, if a change to the value of the dependent scan parameter is not possible under said constraint.

5. A method as claimed in claim 1 comprising determining said suggested change as an adjustment and/or a change to a value of scan parameter in said scan sequence that has previously been adjusted or changed, before selecting said at least one scan parameter to be set.

6. A magnetic resonance (MR) apparatus comprising:
an MR data acquisition scanner;
a control computer configured to operate said MR data acquisition scanner according to a scan sequence comprising a plurality of scan parameters;
said control computer being configured to receive a manual selection entry into said control computer that designates said at least one scan parameter to be set;
said control computer being configured to receive a further entry into said control computer that sets and/or changes a value of the at least one selected scan parameter;
said control computer being configured to determine at least one dependent scan parameter in said scan sequence that has a value which, due to a change of the value of the selected scan parameter to be set, would undergo a change under a constraint that existing image-acquisition criteria are retained;
said computer being configured to determine a suggested change to the value of said at least one dependent scan parameter;
a display in communication with said computer; and
said control computer being configured to display said at least one dependent scan parameter and the suggested change at said display.

7. A non-transitory, computer-readable data storage medium encoded with programming instructions, said storage medium being loaded into a computer of a magnetic resonance (MR) apparatus that comprises an MR data acquisition scanner, said computer being configured to operate the MR data acquisition scanner in order to conduct a magnetic resonance examination by executing a scan sequence comprising a plurality of scan parameters, said programming instructions causing said computer to:
receive a manual selection entry that designates said at least one scan parameter to be set;
receive a further entry that sets and/or changes a value of the at least one selected scan parameter;
determine at least one dependent scan parameter in said scan sequence that has a value which, due to a change of the value of the selected scan parameter to be set, would undergo a change under a constraint that existing image-acquisition criteria are retained;
determine a suggested change to the value of said at least one dependent scan parameter; and
at a display in communication with said computer, display said at least one dependent scan parameter and the suggested change.
